(19) United States
(12) Patent Application Publication
(10) Pub. No.: US 2016/0051289 A1
(43) Pub. Date: Feb. 25, 2016

(54) SCREW RETENTION CAP FOR USE IN SPINE SURGERY

(71) Applicant: Spineology Inc., St. Paul, MN (US)

(72) Inventors: Joe Gleason, Eagan, MN (US); Natalie Kupetsky, St. Paul, MN (US)

(21) Appl. No.: 14/832,992

(22) Filed: Aug. 21, 2015

Related U.S. Application Data
(60) Provisional application No. 62/040,408, filed on Aug. 21, 2014.

(51) Int. Cl. A61B 17/70

(52) U.S. Cl. CPC A61B 17/7037; A61B 17/7032; A61B 2017/00526

(57) ABSTRACT

A retaining cap includes circular projections positioned on the outside of a circumferential surface of a main body of the cap. The projections define a diameter for the cap that is larger than the diameter of the main body of the cap. The retaining cap is joined with the tulip in an assembly by positioning the cap coaxially with the tulip body. The cap is then slid into tulip body with the projections aligned with respective half circle entry slots of tulip body. The cap is the rotated axially to a degree that the tulip's diameter is reduced to cause interference between the retaining cap projections and the tulip. The retaining cap can be rotated past the interfering diameter and into a home position created by cross holes in the tulip body where the cap can be removed from the tulip.

### PRIORITY

This application claims the benefit of U.S. Provisional Application Ser. No. 62/040,408, filed on Aug. 21, 2014, which is hereby incorporated herein by reference in its entirety.

### FIELD

The present invention generally relates to screw retention caps. More particularly, the present invention relates to a pedicle screw retention cap for use in spine surgery.

### BACKGROUND

Pedicle screw systems provide adjunct fixation to the posterior of the spine when used in combination with interbody fusion devices placed in the disc space of the spine for spinal fusion surgeries.

The primary components of a pedicle screw system are: a screw assembly, a spinal rod, and a locking screw. The screw assembly generally comprises a screw with a spherical ball head, a tulip body, and a retaining cap, and is provided to the user as an assembly.

At manufacture, the screw is loaded into the tulip body, positioned into a saddle retaining the screw ball and allowing it to rotate. The retaining cap is then assembled into the tulip body and retained in position by various assembly methodologies.

The function of the retaining cap is to prevent screw disassembly from the tulip body during implantation while allowing the screw to pivot around the screw ball head.

After the screw is implanted, the spinal rod is loaded and the set screw installed, tightening the set screw drives the rod into the retaining cap, which entraps and compresses the retaining cap, screw ball head, and tulip body together. Thus, locking the position of the screw, tulip and rod assembly from rotation and linear slippage.

However, there remains a need for improved retaining caps and screw retaining methods.

### SUMMARY

In one embodiment, a retaining cap allows simple assembly and installation of a screw used in spinal surgery. The retaining cap may include high push out forces along the axis of the screw to prevent unintended disassembly during insertion and prior to lock down.

Disclosed is a retaining cap that includes one or more circular projections positioned on the outside of a circumferential surface of a main body of the cap. The projections define a diameter for the cap that is larger than the diameter of the main body of the cap. The retaining cap is joined with the tulip in an assembly by positioning the cap coaxially with the tulip body. The cap is then slid into tulip body with the projections aligned with respective half circle entry slots of tulip body. The cap is the rotated axially to a degree that the tulip's diameter is reduced to cause interference between the retaining cap projections and the tulip. The retaining cap can be rotated past the interfering diameter and into a home position created by cross holes in the tulip body where the cap can be removed from the tulip.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 is a perspective assembly view of a cap and tulip according to an embodiment of the present invention.

FIG. 2 is a top view of a screw retention cap according to an embodiment of the present invention.

FIG. 3 is a side view of a screw retention cap according to an embodiment of the present invention.

FIG. 4 is a perspective view of a screw retention cap disposed in a tulip according to an embodiment of the present invention.

FIG. 5 is a perspective view of a screw retention cap disposed in a tulip according to an embodiment of the present invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof. However, these embodiments are not intended to limit the present invention to any specific example, embodiment, environment, applications or particular implementations described in these embodiments. Therefore, description of these embodiments is only for purpose of illustration rather than to limit the present invention. It should be appreciated that, in the following embodiments and the attached drawings, elements unrelated to the present invention are omitted from depiction; and dimensional relationships among individual elements in the attached drawings are illustrated only for ease of understanding, but not to limit the actual scale.

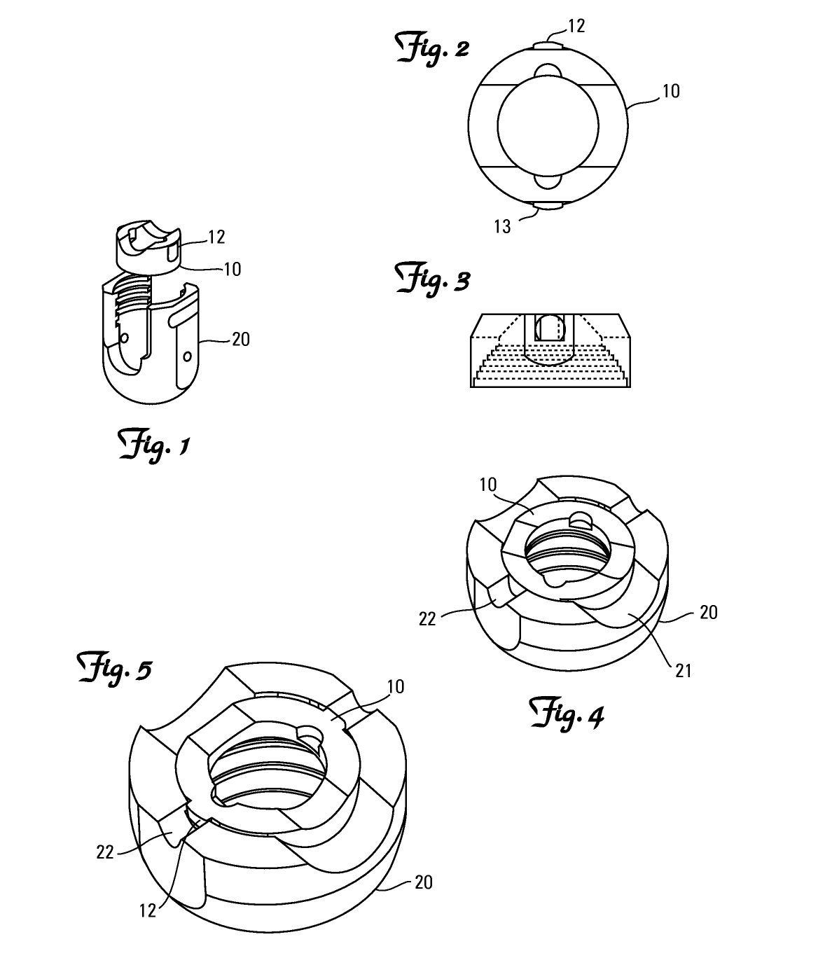

As is depicted in FIGS. 1-5, retaining cap 10 includes circular projections 12 positioned on the outside circumferential surface of body main body of the cap 10. Thus, the projections define a diameter for the cap that is larger than the diameter of the main body of the cap.

The projections can be circular when viewed from the side as shown in FIG. 3, or they can be any other projecting shape, including polygonal, oval, etc. or another complex shape.

The projections 12 when viewed from the top as in FIG. 2 define a leading edge and a trailing edge. The projections 12 can include a chamfered leading edge 13 as shown in FIG. 2.

The number of projections can vary without departing from the scope of the invention (e.g. 1, 2, 3 4 or more). FIGS. 1-5 depict one preferred embodiment, where there are two projections dispose rotationally opposite from one another (i.e. 180 degrees offset). The rotational spacing of the projections can be varied as well.

The tulip body 20 includes opposed half circle entry slots 21 matching the number of projections 12 of the cap 10. The entry slots 21 define a diameter of the tulip matching the effective diameter of retaining cap projections 12. The entry slots are semicircular in the figures, but can have different shapes without departing from the scope of the invention.

The retaining cap 10 is joined with the tulip 20 in an assembly by positioning the cap 10 coaxially with the tulip body 20. The cap 10 is then slid into tulip body 20 until the projections 12 align with the half circle entry slots 21 of tulip body 20. The cap 10 is the rotated axially to a degree that the tulip's diameter is reduced such as in FIG. 4, thereby causing interference between the retaining cap projections 12 and the tulip.

Beyond half circle entry slots 21, retaining cap 10 may be prevented from further rotation by the interference of retaining cap projections 12 and a cylindrical wall of tulip 20. The interference disallows counter rotation without exerting more counter clockwise force than the installation clockwise force due to the lack of the leading chamfer on the back side of retaining cap protrusions 12.

Retaining cap 10 can be rotated in use past the interfering diameter and into a home position created by cross holes 22 in tulip body 20. Then the cap 10 can be removed.

In use, there is virtually no rotation forces on retaining cap 10 which would act to unlock it rotationally. By using the half circle screw in installation method, a larger design interference is achieved by retaining cap protrusions 12 and a cross hole tulip body 20. This greater interference develops significantly higher push out forces axially reducing or eliminating the possibility of inadvertent disassembly of the Screw/Tulip body/Retaining cap prior to installation of the spinal rod and locking set screw.

The entirety of the disclosure of U.S. Pub. Pat. App. No. 2013/0110124 is hereby incorporated in its entirety herein as part of this application.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached hereto.

What is claimed is:

1. A screw retention cap system for use in spine surgery, the system comprising:
a tulip head defining a plurality of entry slots; and
a retaining cap including one or more circular projections positioned on the outside of a circumferential surface of a main body of the cap, wherein the projections define a diameter for the cap that is larger than a diameter of the main body of the cap, and wherein the projections on the cap are alignable with a respective entry slots in the tulip head when the cap is coaxially located with respect to the tulip.

2. The system of claim 1, wherein the projections each define a chamfered leading edge and an opposing trailing edge.

3. A method of retaining a screw in a tulip body for use in spinal surgery; the method comprising:
joining a retaining cap with the tulip by positioning the cap coaxially with the tulip body;
rotating the cap axially with respect to the into tulip body such that each of a plurality of radial projections of the cap are aligned with a respective each of a plurality of entry slots defined in the tulip body;
rotating the cap axially further with respect to the tulip body to a degree that an inner diameter of the tulip is reduced to cause interference between the retaining cap projections and the tulip.

4. The method of claim 3, further comprising rotating the cap with respect to the tulip body past the interfering diameter and into a home position created by a plurality of cross holes defined in the tulip body where the cap can be removed from the tulip.
